(19) United States
(12) Patent Application Publication
(10) Pub. No.: US 2016/0038651 A1
(43) Pub. Date: Feb. 11, 2016

(54) COMPOUNDS AND COMPOSITIONS FOR DRUG RELEASE

(71) Applicant: INTERFACE BIOLOGICS, INC., Toronto (CA)

(72) Inventors: J. Paul SANTERRE, Whitby (CA); Roseita ESFAND, Mississauga (CA)

(21) Appl. No.: 14/776,903

(22) PCT Filed: Mar. 17, 2014
(86) PCT No.: PCT/CA2014/050284

§ 371 Date: Sep. 15, 2015

Related U.S. Application Data
(60) Provisional application No. 61/799,859, filed on Mar. 15, 2013.

(51) Int. Cl. A61L 31/08; A61L 29/16; A61L 29/08; A61L 31/16; A61L 31/06

(52) U.S. Cl. CPC A61L 31/08; A61L 31/16; A61L 31/06; A61L 29/08; A61L 29/16; A61L 2420/02; A61L 2300/406; A61L 2300/80

(57) ABSTRACT

The invention relates to compounds that include biologically active agents (e.g., compounds according to any of formulas (I) and (I-A) that can be used for effective drug release, e.g., as coatings for medical devices. Use of these compounds in the coating of surfaces can allow for long-term drug release as well as imparting uniform coatings with little phase separation compared to, e.g., the parent biologically active agent.

### CROSS-REFERENCE TO RELATED APPLICATIONS

This application claims the benefit of U.S. Provisional Application No. 61/799,859, filed Mar. 15, 2013, which is hereby incorporated by reference in its entirety.

### FIELD OF THE INVENTION

This invention relates to compounds that include biologically active agents that can be used for effective drug release, e.g., as coatings for medical devices.

### BACKGROUND OF THE INVENTION

The appropriate biological response to the surface of a device is crucial for biocompatibility. The coating of a medical device using, e.g., organic compositions, can also serve as a repository for delivery of a biologically active agent. A coating that is used to control release of the drug must be free of impurities that trigger adverse biological responses (i.e., biologically inert), must produce the desired release profile, and must not adversely affect the mechanical properties required of the medical device. Further, when the active agent is a pharmaceutical drug, it is often desirable to release the drug locally from the medical device over an extended period of time.

Systems for kinetically controlled direct drug delivery can employ a polymer that includes a biologically active agent. For example, when the agent is part of the polymer backbone, it may be released as the polymer enzymatically degrades or disintegrates in the body. Drug release by such polymers, however, may be complicated by release of other organic entities, including various biologically active species resulting from incomplete hydrolysis. Alternatively, biologically active agents can be simply mixed with a polymer platform in a suitable solvent system. The biologically active agent is then released by particle dissolution or diffusion (when the non-bioerodable matrices are used) or during polymer breakdown (when a biodegradable polymer is used). In these systems the polymer coating will become part of the device design. Mixing lowers the entropy and this can result in phase separation throughout the bulk polymer, compromising the physical/mechanical properties of the polymeric coating. In addition the presence, stability, and uniform distribution of the drug throughout the polymeric coating can compromise the device performance (e.g., orthopedic devices).

In view of the potential drawbacks to current strategies for drug release by, e.g., coated devices, there exists a need for drug delivery platforms which provide for delivery of biologically active agents with a defined profile of release. The present invention addresses these problems and offers advantages over the current technology.

### SUMMARY OF THE INVENTION

In a first aspect, the invention features an article that includes a coated surface, where said coated surface includes a compound having a structure according to formula (I):

Bio¹-Link¹-(Bio²-R¹)ₘ (I),

or a pharmaceutically acceptable salt thereof, where

Bio¹ is formed from a biologically active agent;

m is 1, 2, 3, 4, or 5;

each Bio² is absent or independently formed from a biologically active agent, and where each Bio², when present, includes a covalent bond to Link¹;

R¹ is present only when Bio² is absent and is a terminal group selected from the group consisting of H, OH, optionally substituted C1-C6 alkyl, and optionally substituted C1-C6 alkoxy;

Link¹ is an oligomeric organic, organosilicon, or organosulfone segment having a molecular weight between 60 and 2000 Daltons.

In some embodiments, the compound has a structure according to formula (I-A),

Bio¹-Link¹-Bio²-R¹ (I-A),

or a pharmaceutically acceptable salt thereof, where

Bio¹ is formed from a biologically active agent;

Bio² is absent or formed from a biologically active agent;

R¹, when present, is H, OH, optionally substituted C1-C6 alkyl, or optionally substituted C1-C6 alkoxy; and

Link¹ is an oligomeric organic, organosilicon, or organosulfone segment having a molecular weight between 60 and 2000 Daltons.

In some embodiments, Bio² is absent.

In other embodiments, Bio² is present.

In certain embodiments, Bio¹ and Bio² are formed from biologically active agents that have the same structure.

In still other embodiments, Bio¹ and Bio² are formed from biologically active agents that have different structures.

In further embodiments, each Bio¹ and Bio², when present, has a molecular weight ranging from 100 to 1000, from 200 to 1000, from 200 to 900, from 200 to 800, from 200 to 700, from 200 to 600, from 200 to 500, or from 200 to 400 Daltons.

In still other embodiments, each Bio¹ and Bio², when present, is formed from a biologically active agent selected from the group consisting of: anti-inflammatory agents, anti-thrombotic agents; anti-oxidant agents, anti-coagulant agents, anti-microbial agents, anti-proliferative agents, cell receptor ligands, and bio-adhesive molecules.

In some embodiments, one or both of Bio¹ and Bio², when present, is formed from an anti-microbial agent.

In other embodiments, one or both of Bio¹ and Bio², when present, is independently, is formed from an antibiotic (e.g., fluoroquinolone antibiotics selected from the group consisting of: norfloxancin, oflxacin, ciprofloxacin, levofloxacin, moxifloxacin, and gatifloxacin). In certain embodiments, the antibiotic is ciprofloxacin.

In still other embodiments, one or both of Bio¹ and Bio² is a protein or a peptide.

In certain embodiments, Link¹ has a molecular weight between 60 and 700 Daltons.

In other embodiments, Link¹ is formed from a diol, a diamine, or an α,ω-aminoalcohol.

In particular embodiments, Link¹ is formed from a diol.

In still other embodiments, Link¹ is formed from a polyethylene oxide having terminal amino or hydroxyl groups, and where Link1 includes 1-3, 1-5, 1-10, or 1-20 ethylene oxide repeating units.

In some embodiments, Link¹ is formed from a compound selected from the group consisting of: ethylene glycol; butane diol; hexane diol; hexamethylene diol; 1,5-pentanediol; 2,2-dimethyl-1,3 propanediol; 1,4-cyclohexane diol; 1,4-cyclohexanedimethanol; tri(ethylene glycol); poly(ethylene glycol), where the molecular weight is between 100 and 2000 Daltons; poly(ethylene oxide) diamine, where the molecular weight is between 100 and 2000 Daltons; lysine esters; silicone diols; silicone diamines; polyether diols; polyether diamines; carbonate diols; carbonate diamines; dihydroxy vinyl derivatives; dihydroxydiphenylsulfone; ethylene diamine; hexamethylene diamine 1,2-diamino-2-methylpropane; 3,3-diamino-n-methyldipropylamine; 1,4-diaminobutane; 1,7-diaminoheptane; and 1,8-diaminooctane.

In particular embodiments, Link¹ is formed from tri(ethylene glycol).

In still other embodiments, Link¹ is formed from a dicarboxylic compound or a diisocyanate.

In further embodiments, Bio² is absent, and Link¹ is formed from a monoalcohol or a monoamine.

In certain embodiments, m is 1, Bio¹ and Bio² are both formed from ciprofloxacin, and Link¹ is formed from tri(ethylene glycol).

In still other embodiments, m is 1, Bio¹ is formed from ciprofloxacin, Bio² is absent, and Link¹ is formed from tri(ethylene glycol).

In particular embodiments, the coating includes a second compound having a structure according to formula (I) or formula (I-A), where each Bio¹, Link¹, and Bio² is as defined in any embodiment, or a combination of embodiments, described herein.

In still other embodiments, the coating is substantially free of any biologically active agent used to form Bio¹ and/or Bio² where the biologically active agent is not included in a compound according to formula (I) or formula (I-A).

In further embodiments, the coating further includes free biologically active agent, where the mole ratio of the compound according to formula (I) to the free biologically active agent is from 0.1:1 to 1:0.1.

In certain embodiments, compound according to formula (I) or formula (I-A) has reduced biological activity compared to the biologically active agent used to form Bio¹ and/or Bio².

In still other embodiments, the compound according to formula (I) or formula (I-A) has 0%-20% of the biological activity of the biologically active agent used to form Bio¹ and/or Bio².

In some embodiments, the coating includes a pharmaceutically acceptable salt of the compound according to formula (I) or formula (I-A).

In further embodiments, the pharmaceutically acceptable salt is the trifluoroacetate or the hydrochloride salt.

In certain embodiments, the article is a filter, film, fiber, sheet, or an implantable medical device.

In particular embodiments, the implantable device is selected from the group consisting of: prostheses pacemakers, electrical leads, defibrillators, artificial hearts, ventricular assist devices, anatomical reconstruction prostheses, artificial heart valves, heart valve stents, pericardial patches, surgical patches, coronary stents, vascular grafts, vascular and structural stents, vascular or cardiovascular shunts, biological conduits, pledges, sutures, annuloplasty rings, stents, staples, valved grafts, dermal grafts for wound healing, orthopedic spinal implants, orthopedic devices, ophthalmic implants, intrauterine devices, stents, maxial facial reconstruction plating, dental implants, intraocular lenses, clips, sternal wires, bone, skin, ligaments, sutures, hernia mesh, tendons, and combinations thereof

In other embodiments, the article is a percutaneous device selected from: catheters, cannulas, drainage tubes, and surgical instruments, or the article is a cutaneous device selected from burn dressings, wound dressings and dental hardware.

In some embodiments, the surgical instrument is selected from: forceps, retractors, needles, gloves, and catheter cuffs.

In other embodiments, the article is a catheter cuff.

In still other embodiments, the coating has a thickness between 0.5 to 120 μM.

In some embodiments, the article includes a fibrous polymer matrix that includes one or more compounds according to formula (I) and/or formula (I-A).

In particular embodiments, the article includes an admixture that includes a two or more compounds according to formula (I) and/or formula (I-A).

In certain embodiments, polymer matrix is formed from a biodegradable polymer.

In other embodiments, polymer is polylactic acid or polycaprolactone.

In some embodiments, polymer matrix is formed from a nonbiodegradable polymer.

In still other embodiments, the polymer is poly(ethylene terephthalate).

In certain embodiments, article is a catheter cuff.

In still other embodiments, the catheter cuff is a vascular access catheter cuff.

In further embodiments, the article is an orthopedic device.

In still other embodiments, orthopedic device is a wire, pin, rod, nail, screw, disk, plate, bracket, or splint.

In some embodiments, the ophthalmic implant is a punctal plug.

In particular embodiments, the article contains two or more compounds having a structure according to formula (I). In certain embodiments, the article contains two or more compounds having a structure according to formula (I-A). In other embodiments, the article contains one or more compounds having a structure according to formula (I) and one or more compounds having a structure according to formula (I-A).

In a second aspect, the invention features a method of preventing infection in a subject in need thereof, where the method includes implanting a device that includes a coated surface, where said coated surface includes a compound having a structure according to formula (I):

Bio¹-Link¹-(Bio²-R¹)ₘ (I),

or a pharmaceutically acceptable salt thereof, where

Bio¹ is formed from a biologically active agent;

m is 1, 2, 3, 4, or 5;

each Bio² is absent or independently formed from a biologically active agent, and where each

Bio², when present, includes a covalent bond to Link¹;

R¹ is present only when Bio² is absent and is a terminal group selected from the group consisting H, OH, optionally substituted C1-C6 alkyl, and optionally substituted C1-C6 alkoxy;

Link¹ is an oligomeric organic, organosilicon, or organosulfone segment having a molecular weight between 60 and 2000 Daltons.

In certain embodiments, the compound has a structure according to formula (I-A),

Bio¹-Link¹-Bio²-R¹ (I-A),

or a pharmaceutically acceptable salt thereof, where

Bio¹ is formed from a biologically active agent;

Bio² is absent or formed from a biologically active agent;

R¹, when present, is H, OH, optionally substituted C1-C6 alkyl, or optionally substituted C1-C6 alkoxy; and

Link¹ is an oligomeric organic, organosilicon, or organosulfone segment having a molecular weight between 60 and 2000 Daltons.

In particular embodiments, the compound has a structure according to any embodiment described herein for a compound according to formula (I) or formula (I-A), or combination of embodiments thereof.

In a third aspect, the invention features an admixture that includes a base polymer and a compound having a structure according to formula (I),

Bio¹-Link¹-(Bio²-R¹)ₘ (I),

or a pharmaceutically acceptable salt thereof, where

Bio¹ is formed from a biologically active agent;

m is 1, 2, 3, 4, or 5;

each Bio² is absent or independently formed from a biologically active agent, and where each

Bio², when present, includes a covalent bond to Link¹;

R¹ is present only when Bio² is absent and is a terminal group selected from the group consisting of H, OH, optionally substituted C1-C6 alkyl, and optionally substituted C1-C6 alkoxy;

Link¹ is an oligomeric organic, organosilicon, or organosulfone segment having a molecular weight between 60 and 2000 Daltons.

In some embodiments, the compound has a structure according to formula (I-A),

Bio¹-Link¹-Bio²-R¹ (I-A),

or a pharmaceutically acceptable salt thereof, where

Bio¹ is formed from a biologically active agent;

Bio² is absent or formed from a biologically active agent;

R¹, when present, is H, OH, optionally substituted C1-C6 alkyl, or optionally substituted C1-C6 alkoxy; and

Link¹ is an oligomeric organic, organosilicon, or organosulfone segment having a molecular weight between 60 and 2000 Daltons.

In particular embodiments, the compound has a structure according to any embodiment described herein for a compound according to formula (I) or formula (I-A), or combination of embodiments thereof.

In certain embodiments, the admixture is a polymer matrix.

In a fourth embodiment, the invention features a method for coating a surface, where the composition includes:
(a) a compound having a structure according to formula (I),

Bio¹-Link¹-(Bio²-R¹)ₘ (I),
or a pharmaceutically acceptable salt thereof, where
Bio¹ is formed from a biologically active agent;
m is 1, 2, 3, 4, or 5;
each Bio² is absent or independently formed from a biologically active agent, and where each Bio², when present, includes a covalent bond to Link¹;
R¹ is present only when Bio² is absent and is a terminal group selected from the group consisting of H, OH, optionally substituted C1-C6 alkyl, and optionally substituted C1-C6 alkoxy;
Link¹ is an oligomeric organic, organosilicon, or organosulfone segment having a molecular weight between 60 and 2000 Daltons
and
(b) a suitable medium in which the compound of (a) is soluble; and

where said composition is substantially free of any biologically active agent used to form Bio¹ and/or Bio² where the biologically active agent is not included in a compound according to formula (I).

In some embodiments, the compound has a structure according to formula (I-A),

Bio¹-Link¹-Bio²-R¹ (I-A),

or a pharmaceutically acceptable salt thereof, where

Bio¹ is formed from a biologically active agent;

Bio² is absent or formed from a biologically active agent;

R¹, when present, is H, OH, optionally substituted C1-C6 alkyl, or optionally substituted C1-C6 alkoxy; and

Link¹ is an oligomeric organic, organosilicon, or organosulfone segment having a molecular weight between 60 and 2000 Daltons.

In particular embodiments, the compound has a structure according to any embodiment described herein for a compound according to formula (I) or formula (I-A), or combination of embodiments thereof.

In further embodiments, the component of (b) is an organic solvent or aqueous solvent.

In certain embodiments, the polar organic solvent is tetrahydrofuran, N,N-dimethylformamide, diethylamine, chloroform, methyl t-butyl ether, toluene, benzene, ether, p-xylene, carbon disulfide, carbon tetrachloride, cyclohexane, pentane, hexane, heptane, dioxane, ethylacetate, dimethoxyethane, ethyl benzoate, anisol, chlorobenzene, pyridine, acetone, dimethylsulfoxide, acetonitrile, ethanol, n-propanol, toluene, methanol, water, or benzyl alcohol.

In still other embodiments, the concentration of (a) is between 0.05-150 mg/mL.

In certain embodiments of any aspect of the invention, the article contains two or more compounds having a structure according to formula (I) and/or formula (I-A). In other embodiments, Bio¹ of the compound of formula (I) or (IA) is ciprofloxacin. In yet other embodiments, Bio¹ of the compound of formula (I) or (IA) is hydrocortisone.

In certain embodiment of any aspect of the invention, the molecular weight is a theoretical molecular weight.

By the term “oligomeric segment” is meant a relatively short length of a repeating unit or units, generally less than about 50 monomeric units and molecular weights less than 10,000 but preferably <5000. Oligomeric segments can be selected from the group consisting of polyurethane, polyurea, polyamides, polyalkylene oxide, polycarbonate, polyester, polylactone, polysilicone, polyethersulfone, polyolefin, polyvinyl, polypeptide, polysaccharide; and ether and amine linked segments thereof, or other multifunctional compounds as described herein. The linking segments (e.g., the Link¹ segments) described herein can include oligomeric segments.

Typically, Link (e.g., Link¹) molecules can have molecular weights ranging from 60 to 2000 and preferably 60-700, and have difunctionality to permit coupling of two oligo units. Preferably the Link molecules are synthesized from diamines, diisocyanates, disulfonic acids, dicarboxylic acids, diacid chlorides and dialdehydes. Terminal hydroxyls, amines or carboxylic acids on the oligo molecules can react with diamines to form oligo-amides; react with diisocyanates to form oligo-urethanes, oligo-ureas, oligo-amides; react with disulfonic acids to form oligo-sulfonates, oligo-sulfonamides; react with dicarboxylic acids to form oligo-esters, oligo-amides; react with diacid chlorides to form oligo-esters, oligo-amides; and react with dialdehydes to form oligo-acetal, oligoimines.

The terms “pharmaceutically active agent” and “biologically active agent”, or precursor thereof, refer to a molecule that can be coupled to a Link segment via hydrolysable covalent bonding. Hydrolysable covalent bonds are those that can undergo spontaneous or catalyzed (e.g., enzyme-catalyzed) hydrolytic cleavage under physiological conditions (e.g., mammalian physiological conditions). Non-limiting examples of functional groups containing hydrolysable covalent bonds include: esters, thioesters, amides, thioamides, sulfonamides, sulfinamides, acid anhydrides, imides, imines, phosphate esters, and phosphonate esters. Accordingly, each biologically active agent used to form [Bio¹] and/or [Bio²], includes at least one group selected independently from the group consisting of carbonyl group, amine, phosphonate, phosphate, sulfonate, sulfinate, and a combinations thereof. Thus, the compounds of the invention, when implanted in vivo as part of a coating, undergo hydrolysis of one or more of the groups containing hydrolysable covalent bonds, thereby releasing defined degradation products consisting of biological, pharmaceutical, and/or biocompatible components. The molecule must have some specific and intended pharmaceutical or biological action. Typically the [Bio] unit has a molecular weight ranging from 40 to 2000 for pharmaceuticals but may be higher for biopharmaceuticals depending on the structure of the molecule. Preferably, the Bio unit is selected from the group of anti-inflammatory, anti-oxidant, anti-coagulant, anti-microbial (including fluoroquinolones), antimicrobial enzyme (including lysostaphin), cell receptor ligands and bio-adhesive molecules, specifically oligo-peptides and oligo-saccharides, oligonucleic acid sequences for DNA and gene sequence bonding, and phospholipid head groups to provide cell membrane mimics.

The term “pharmaceutically acceptable salt” as used herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, S. M. Berge et al. describe pharmaceutically acceptable salts in detail in J. Pharm. Sci. 66:1-19, 1977. The salts can be prepared in situ during the final isolation and purification of the compounds of the invention or separately by reacting the free base group with a suitable organic acid. Representative acid addition salts include acetate, adipate, alginate, ascorbate, aspartate, benzenesulfonate, benzoate, bisulfate, borate, butyrate, camphorate, camphersulfonate, carbonate, chloride, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The term “theoretical molecular weight” in this specification is the term given to the absolute molecular weight that would result from the reaction of the reagents utilized to synthesize any given bioactive polymers. As is well known in the art, the actual measurement of the absolute molecular weight is complicated by physical limitations in

the molecular weight analysis of polymers using gel permeation chromatography methods. Hence, a polystyrene equivalent molecular weight is reported for gel permeation chromatography measurements. Since many biologically active compounds absorb light in the UV region, the gel permeation chromatography technique also provides a method to detect the distribution of biologically active compound coupled within polymer chains.

### BRIEF DESCRIPTION OF THE DRAWINGS

FIGS. 1-A and 1-B show the SEM analysis of coated Dacron meshes and Hernia meshes coated with Compound 2 and Compound 3 in DMF, which showed a smooth coating with limited webbing. Dacron mesh coated with compound 2 and Chlorohexidine shows a smooth and uniform coating. Dacron meshes coated with Compound 2 or Compound 3 are shown in FIG. 1-A. Hernia meshes (control) and those coated with Compound 2 or Compound 2 plus chlorhexidine are shown in FIG. 1-B.

FIG. 2 shows the SEM and Confocal light microscopy images showing ciprofloxacin.HCl vs compound 2 distribution in scaffold fibers. Electro-spun material (polyurethane with Ciprofloxacin or compound 2) shows smooth and uniform coating with compound 2 vs the drug alone (electro-spun). SEM (Top images of Cipro.HCl (left) and Compound 2 in polymer admixture (right)), and Confocal light microscopy images (bottom panel) (A) Control Fiber, (B) Compound 2 and polymer admixture fiber, (C) Compound 2 and polymer admixture fiber and (D) Ciprofloxacin HCl and polymer admixture fiber. Aggregated drug is seen as non-fiber clumps (white arrows) in the polymer fibers containing drug alone. Scale bars=50 μm.

FIG. 3 shows stainless steel coupons and orthopedic screws that were dipped once for thirty seconds in either a 10 mg/mL solution of Compound 2 in organic solvent, Compound 3 in organic solvent, or ciprofloxacin hydrochloride in organic solvent, or DMF (control). Coupons with ciprofloxacin hydrochloride had a white uneven coating, while those coated with Compounds 2 and 3 were clear.

FIG. 4 shows gel matrices that include ciprofloxacin HCl, Compound 2, and Compound 3 that were formed from a 3% alginate solution in water and which were crosslinked using CaSO₄. Gels with Compounds 2 and 3 were clear similar to alginate alone while gels with ciprofloxacin HCl were opaque.

FIG. 5 shows studies relating to the compatibility of ciprofloxacin or Compound 2 as additives in various base polymers. Films prepared by blending base polymer and Compound 2 demonstrated a homogenous morphology.

FIG. 6 relates to drug release from Compound 2 in PBS at 37° C. After 28 days, ˜8% total drug was released from Compound 2, demonstrating slow and sustained release under these conditions.

FIG. 7 relates to drug release from Compound 3 in PBS at 37° C. A linear increase in the drug concentration was observed with time out to at least 28 days.

### DETAILED DESCRIPTION OF THE INVENTION

This invention relates to compounds that include biologically active agents that can be used for effective drug release, e.g., as coatings for medical devices. The biologically active agents include biologically active agents linked via oligomeric segments. The advantages of the invention include improved thermodynamic compatibility of drugs with processing agents, thereby providing: (i) the ability to form uniform coatings on polymeric and metallic surfaces without the complications of phase separation, and drug crystallization, When combined with other coating materials, such as base polymers; (ii) uniform distribution of drugs throughout the coatings when the compounds are used in admixture with polymers (e.g., base polymers) to form films, fibers, and extruded articles; (iii) localization of drugs at therapeutic concentrations; (iv) stability of drugs under processing and storage conditions; and (v) formulation in a stable liquid phase which can be used for further processing. An article of the invention may include a coated surface containing one or more (e.g., two or more) compounds of formula (I) or one or more (e.g., two or more) compounds of formula (I-A). Alternatively, an article of the invention may include a coated surface containing one or more (e.g., two or more) compound of formula (I) and one or more (e.g., two or more) compound of formula (I-A).

### Oligomeric Segments

The compounds described herein include a LINK¹ moiety, which is an oligomeric segment. By “oligomeric segment” or “Oligo” is meant a relatively short length of a repeating unit or units, generally fewer than about 50 monomeric units and molecular weights between 60 and 2000 Daltons. The LINK¹ moiety has multi-functionality, but preferably di-functionality, to permit covalent bond formation to, e.g., a biologically active agent such as Bio¹ and/or Bio². The coupling segments can be synthesized from the groups of precursor monomers selected from diols, diamines and/or a compounds containing both amine and hydroxyl groups. Precursors that can be incorporated into coupling segments include, without limitation, ethylene glycol, butane diol, hexane diol, hexamethylene diol, 1,5-pentanediol, 2,2-dimethyl-1,3 propanediol, 1,4-cyclohexane diol, 1,4-cyclohexanedimethanol, tri(ethylene glycol), poly(ethylene glycol), poly(ethylene oxide) diamine, lysine esters, silicone diols and diamines, polyether diols and diamines, carbonate diols and diamines, dihydroxy vinyl derivatives, dihydroxy diphenylsulfone, ethylene diamine, hexamethylene diamine, 1,2-diamino-2 methylpropane, 3,3-diamino-n-methyldipropylamine, 1,4-diaminobutane, 1,7 diaminoheptane, 2,2,4-trimethylhexamethylene diamine, and 1,8-diaminooctane. Alternatively, Link¹ can be formed from a moiety that is a bifunctional electrophile such as diisocyanates, dicarboxylates, diesters, and dicarbonates.

### Biologically Active Agents

Preferred Bio components include but are not limited to the following categories and examples: Anti-inflammatory: non-steroidal-Oxaceprol, steroidal Enoxolone; antithrombotic: Tirofiban, Lotrafiban; anti-coagulant: heparin; anti-proliferation: acivicin and alkeren; anti-microbial: fluoroquinolones such as norfloxancin, ciprofloxacin, sparfloxacin and trovafloxacin and other fluoroquinolones, and antiproliferative agents such as paclitaxel. Exemplary, non-limiting Bio components are provided in Tables 1 and 2.

TABLE 1
Exemplary Pharmaceutical Molecules Used for the Synthesis of Compounds of Formula (I)

| Pharmaceuticals | Function | Chemical structures |
|---|---|---|
| Norfloxacin | Antimicrobial | 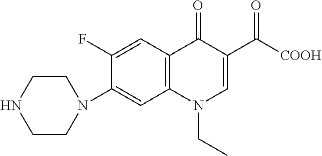 [SMILES: CCn1cc(C(=O)C(=O)O)c(=O)c2cc(F)c(N3CCNCC3)cc21] |
| Ciprofloxacin | Antimicrobial | 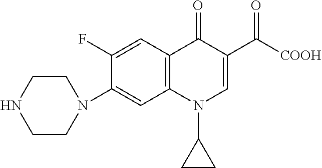 [SMILES: O=C(O)C(=O)c1cn(C2CC2)c2cc(N3CCNCC3)c(F)cc2c1=O] |
| Amfenac | Antiinflammatory | 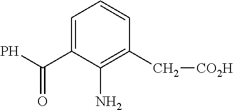 |
| Aceclofenac | Antiinflammatory | 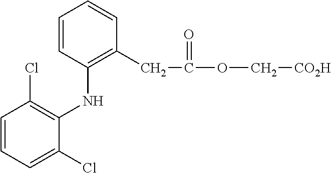 [SMILES: O=C(O)COC(=O)Cc1ccccc1Nc1c(Cl)cccc1Cl] |
| Oxaceprol | Antiinflammatory | 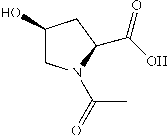 |
| Enoxolone | Antiinflammatory | 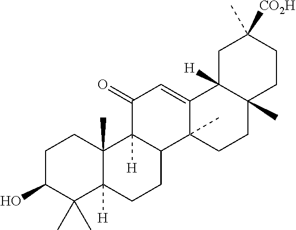 |
| Hydrocortisone | Antiinflammatory | 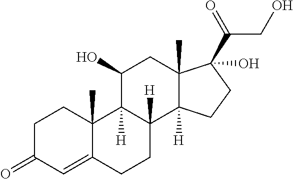 |
| Ibuprofen | Antiinflammatory | 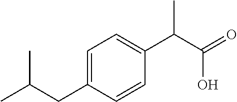 |
| Bromofenac | Antithrombic | 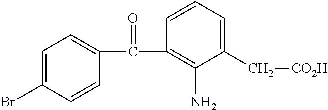 [SMILES: Nc1c(CC(=O)O)cccc1C(=O)c1ccc(Br)cc1] |
| Tirofiban | Antithrombic | 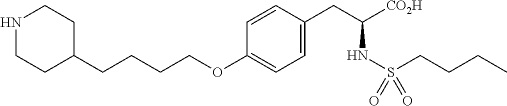 [SMILES: CCCCS(=O)(=O)N[C@@H](Cc1ccc(OCCCCC2CCNCC2)cc1)C(=O)O] |
| Tirofiban | Antithrombic | 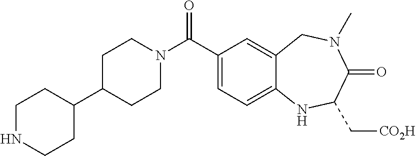 [SMILES: CN1Cc2cc(C(=O)N3CCC(C4CCNCC4)CC3)ccc2N[C@@H](CC(=O)O)C1=O] |
| Acivicin | Antiproliferation | 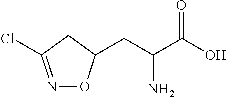 [SMILES: NC(CC1CC(Cl)=NO1)C(=O)O] |
| Alkeren | Antiproliferation | 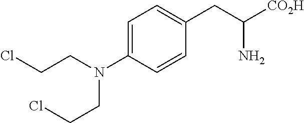 [SMILES: NC(Cc1ccc(N(CCCl)CCCl)cc1)C(=O)O] |

Antibacterials can be of particular use, and exemplary antibacterials that can be used in the compounds and coatings described herein include the following:

TABLE 2

| Name | Application |
|---|---|
| Norfloxacin | uncomplicated urinary tract |
| | infections |
| Ofloxacin | opthalmology |
| | urinary tract and catheter-related |
| | infections |
| | gastroenteritis |
| Ciprofloxacin | opthalmology |
| | urinary tract and catheter-related |
| | infections |
| | gastroenteritis |
| Levofloxacin | ophthalmology |
| | urinary tract infection |
| | kidney infection |
| | prostatis |
| Moxifloxacin | respiratory tract infection |
| | tuberculosis |
| | endocarditis |
| | skin infection |
| | ophthalmology |
| Gatifloxacin | ophthalmology |

Still other biologically active agents include a substantially purified peptide or protein. Proteins are generally defined as consisting of 100 amino acid residues or more; peptides are less than 100 amino acid residues. Unless otherwise stated, the term protein, as used herein, refers to both proteins and peptides. The proteins may be produced, for example, by isolation from natural sources, recombinantly, or through peptide synthesis. Examples include growth hormones, such as human growth hormone and bovine growth hormone; enzymes, such as DNase, proteases, urate oxidase, alronidase, alpha galactosidase, and alpha glucosidase; antibodies, such as trastuzumab.

### Combination Therapy

In addition to one or more (e.g., two or more) compounds of the invention (e.g., the compounds of formula (I) or (I-A)), the coated surface of an article of the invention may contain an additional free biologically active agent, e.g., an antibiotic agent. Examples of antibiotic agents include: aminoglycosides, such as amikacin, apramycin, arbekacin, bambermycins, butirosin, dibekacin, dihydrostreptomycin, fortimicin(s), fradiomycin, gentamicin, ispamicin, kanamycin, micronomicin, neomycin, neomycin undecylenate, netilmicin, paromomycin, ribostamycin, sisomicin, spectinomycin, streptomycin, streptonicozid, and tobramycin; amphenicols, such as azidamfenicol, chloramphenicol, chloramphenicol palmirate, chloramphenicol pantothenate, florfenicol, and thiamphenicol; ansamycins, such as rifampin, rifabutin, rifapentine, and rifaximin; β-Lactams, such as amidinocillin, amdinocillin, pivoxil, amoxicillin, ampicillin, aspoxicillin, azidocillin, azlocillin, bacampicillin, benzylpenicillinic acid, benzylpenicillin, carbenicillin, carfecillin, carindacillin, clometocillin, cloxacillin, cyclacillin, dicloxacillin, diphenicillin, epicillin, fenbenicillin, floxicillin, hetacillin, lenampicillin, metampicillin, methicillin, mezlocillin, nafcillin, oxacillin, penamecillin, penethamate hydriodide, penicillin G benethamine, penicillin G benzathine, penicillin G benzhydrylamine, penicillin G calcium, penicillin G hydragamine, penicillin G potassium, penicillin G, procaine, penicillin N, penicillin O, penicillin V, penicillin V benzathine, penicillin V hydrabamine, penimepicycline, phenethicillin, piperacillin, pivapicillin, propicillin, quinacillin, sulbenicillin, talampicillin, temocillin and ticarcillin; carbapenems, such as imipenem; cephalosporins, such as 1-carba (dethia) cephalosporin, cefactor, cefadroxil, cefamandole, cefatrizine, cefazedone, cefazolin, cefixime, cefmenoxime, cefodizime, cefonicid, cefoperazone, ceforanide, cefotaxime, cefotiam, cefpimizole, cefpirimide, cefpodoxime proxetil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin, cephalothin, cephapirin sodium, cephradine, pivcefalexin, cephalothin, cefaclor, cefotetan, cefprozil, loracarbef, cefetamet, and cefepime; cephamycins such as cefbuperazone, cefmetazole, cefminox, cefetan, and cefoxitin; monobactams such as aztreonam, carumonam, and tigemonan; oxacephems such as flomoxef and moxolactam; lincosamides such as clindamycin and lincomycin; macrolides such as azithromycin, carbomycin, clarithromycin, erythromycin(s) and derivatives, josamycin, leucomycins, midecamycins, miokamycin, oleandomycin, primycin, rokitamycin, rosaramicin, roxithromycin, spiramycin and troleandomycin; polypeptides such as amphomycin, bacitracin, capreomycin, colistin, enduracidin, enylomycin, fusafungine, gramicidin(s), gramicidin S, mikamycin, polymyxin, polymyxin β-methanesulfonic acid, pristinamycin, ristocetin, teicoplanin, thiostrepton, tuberactinomycin, tyrocidine, tyrothricin, vancomycin, viomycin(s), virginiamycin and zinc bacitracin; tetracyclines such as spicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, senociclin and tetracycline; and 2,4-diaminopyrimidines such as brodimoprim, tetroxoprim and trimethoprim; nitrofurans such as furaltadone, furazolium, nifuradene, nifuratel, nifurfoline, nifurpirinol, nifurprazine, nifurtoinol and nitrofurantoin; sulfonamides such as acetyl sulfamethoxypyrazine, acetyl sulfisoxazole, azosulfamide, benzylsulfamide, chloramine-β, chloramine-T, dichloramine-T, formosulfathiazole, N₂-formyl-sulfisomidine, N₄-β-D-glucosylsulfanilamide, mafenide, 4′-(methyl-sulfamoyl)sulfanilanilide, p-nitrosulfathiazole, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidochrysoidine, sulfamoxole, sulfanilamide, sulfanilamidomethanesulfonic acid triethanolamine salt, 4-sulfanilamidosalicyclic acid, N₄-sulfanilylsulfanilamide, sulfanilylurea, N-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine and sulfisoxazole; sulfones, such as acedapsone, acediasulfone, acetosulfone, dapsone, diathymosulfone, glucosulfone, solasulfone, succisulfone, sulfanilic acid, p-sulfanilylbenzylamine, p,p′-sulfonyldianiline-N,N′digalactoside, sulfoxone and thiazolsulfone; lipopeptides such as daptomycin; oxazolidones such as linezolid; ketolides such as telithromycin; and miscellaneous antibiotics such as clofoctol, hexedine, magainins, methenamine, methenamine anhydromethylene-citrate, methenamine hippurate, methenamine mandelate, methenamine sulfosalicylate, nitroxoline, squalamine, xibornol, cycloserine, mupirocin, and tuberin.

### Synthesis

Compounds of the invention may be prepared according to methods known in the art. A non-limiting example of a general synthetic procedure for preparing compounds of the invention (e.g., compounds according to formula (I) or formula (I-A)) is provided in Scheme A.

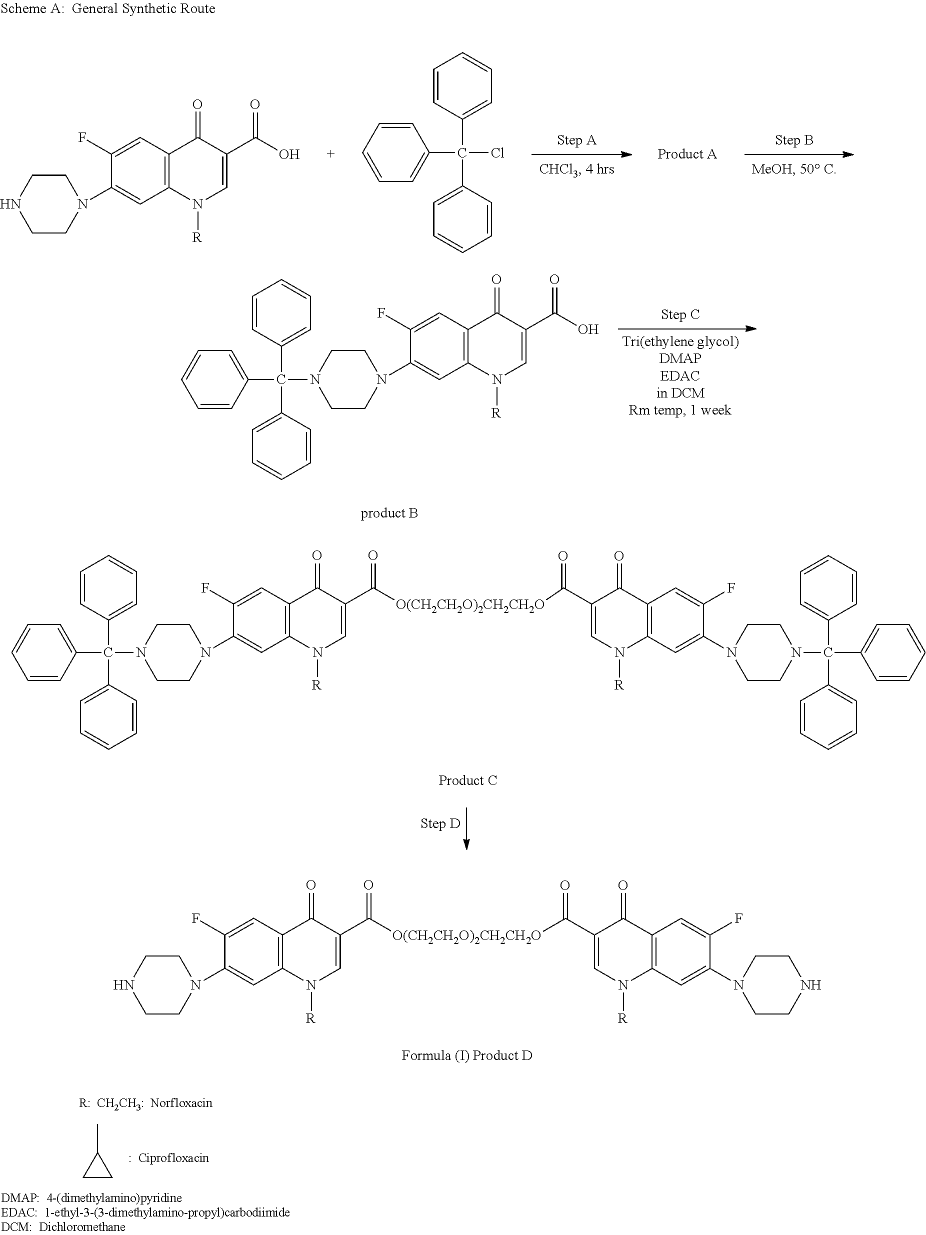

In step A, a biologically active drug, such as norfloxacin or ciprofloxacin (in the form of hydrochloride salt), is protected by a reaction between the biologically active agent and a protecting group precursor, such as trityl halide, in a suitable solvent, such as chloroform. Many other solvents may be needed depending on the solubility of the selected protecting groups and the agents forming the compound of the invention. Suitable trityl halides include trityl chloride and trityl bromide. In step B, the reaction product of step A, such as norfloxacin/ciprofloxacin with both amine and carboxylic acid groups protected with trityl group, is selectively deprotected to yield product B containing free carboxylic acid and N-tritylamine groups. In step C, the purified amine-protected fluroquinolone is coupled to both sides of a diol or diamine (in this example, triethylene glycol is used) containing an appropriate precursor. For example, the purified amine-protected fluroquinolone (Product B) is coupled to a tri(ethylene glycol) in the presence of a suitable coupling agent such as l-ethyl-3-(3-dimethylamino-propyl)carbodiimide herein denoted as EDAC and an appropriate base such as 4-(dimethylamino)pyridine herein denoted as DMAP as a catalyst. Other coupling reagents may include various carbodiimides such as CMC (1-cyclohexyl-3-(2-morpholinoethyl)carbodiimide), DCC (N,N′-dicyclohexyl-carbodiimide), DIC (Diisopropyl carbodiimide) etc, but are not limited to these. In step D, the N-trityl amine groups of the purified product C are deprotected to yield the corresponding desired product. Further synthetic details are provided in the examples.

Admixtures with Base Polymers

In some embodiments, it may be desirable to prepare a blend with a base polymer to produce the requisite mechanical properties, e.g., for a shaped article. Desirably, the polymer of the invention is concentrated within the nm region of the exterior polymer interface and is designed to be thermodynamically compatible with the base polymer to prevent phase separations.

Examples of typical base polymers of use in admixture with the compounds described herein according to the invention, include polyurethanes, polysulfones, polycarbonates, polyesters, polyethylene, polypropylene, polystyrene, polysilicone, poly(acrylonitrile-butadienestyrene}, polyamide, polybutadiene, polyisoprene, polymethylmethacrylate, polyvinyl acetate, polyacrylonitrile, polyvinyl chloride, polyethylene terephtahate, cellulose and other polysacharides. Preferred polymers include polyamides, polyurethanes, polysilicones, polysulfones, polyolefins, polyesters, polyvinyl derivatives, polypeptide derivatives and polysaccharide derivatives. More preferably, in the case of biodegradable base polymers these would include segmented polyurethanes, polyesters, polycarbonates, polysaccharides or polyamides.

In particular, base polymers useful in the blends of the invention can include, without limitation, polyurethane, polysulfones, polycarbonates, polysaccharides, polyesters, polyethylene, polypropylene, polystyrene, poly(acrylonitrile-butadienestyrene), polybutadiene, polyisoprene, styrenebutadiene-styrene block copolymers, styrene-isoprenestyrene block copolymers, poly-R-methylpentene, polyisobutylene, polymethyl-methacrylate, polyvinylacetate-polyacrylonitrile, polyvinyl chloride, polyethyleneterephthalate, cellulose and its esters and derivatives, polyamides, polyester-polyethers, styrene-isoprenes, styrenebutadienes, thermoplastic polyolefins, styrene-saturated olefins, polyester-polyester, ethylene-vinyl acetate ethylene-ethyl acrylate, ionomers, and thermoplastic polydienes.

### Shaped Articles

The compounds described herein can be used as coatings for shaped articles. Any shaped article can be coated with the compounds, compositions, and/or admixtures of the invention. For example, articles suitable for contact with bodily fluids, such as medical can be coated using the compositions described herein. The duration of contact may be short, for example, as with surgical instruments or long term use articles such as implants. The medical devices include, without limitation, catheters, guide wires, vascular stents, micro-particles, electronic leads, probes, sensors, drug depots, transdermal patches, vascular patches, blood bags, orthopedics (e.g., screws and plates), hernia mesh, ophthalmological devices (i.e., punctal plug, contact lenses), vaginal slings, and tubing.

Coatings or admixed compositions according to the invention may be used as a surface covering for an article, or, most preferably, where the polymers or admixtures are of a type capable of being formed into 1) a self-supporting structural body, 2) a film; or 3) a fiber, preferably woven or knit. The composition may comprise a surface or in whole or in part of the article, preferably, a biomedical device or device of general biotechnological use. In the case of the former, the applications may include cardiac assist devices, tissue engineering polymeric scaffolds and related devices, cardiac replacement devices, cardiac septal patches, intra aortic balloons, percutaneous cardiac assist devices, extra-corporeal circuits, A-V fistual, dialysis components (tubing, filters, membranes, etc.), aphoresis units, membrane oxygenator, cardiac by-pass components (tubing, filters, etc.), pericardial sacs, contact lens, cochlear ear implants, sutures, sewing rings, cannulas, contraceptives, syringes, o-rings, bladders, penile implants, drug delivery systems, drainage tubes, pacemaker lead insulators, heart valves, blood bags, coatings for implantable wires, catheters, vascular stents, angioplasty balloons and devices, bandages, heart massage cups, tracheal tubes, mammary implant coatings, artificial ducts, craniofacial and maxillofacial reconstruction applications, ligaments, fallopian tubes. The applications of the latter include the synthesis of bioresorbable polymers used in products that are environmentally friendly (including but not limited to garbage bags, bottles, containers, storage bags and devices, products which could release reagents into the environment to control various biological systems including control of insects, biologically active pollutants, elimination of bacterial or viral agents, promoting health related factors including enhancing the nutritional value of drinking fluids and foods, or various ointments and creams that are applied to biological systems (including humans, animals and other).

The medical device can be an implanted device, percutaneous device, or cutaneous device. Implanted devices include articles that are fully implanted in a patient, i.e., are completely internal. Percutaneous devices include items that penetrate the skin, thereby extending from outside the body into the body. Cutaneous devices are used superficially. Implanted devices include, without limitation, prostheses such as pacemakers, electrical leads such as pacing leads, defibrillarors, artificial hearts, ventricular assist devices, anatomical reconstruction prostheses such as breast implants, artificial heart valves, heart valve stents, pericardial patches, surgical patches, coronary stents, vascular grafts, vascular and structural stents, vascular or cardiovascular shunts, biological conduits, pledges, sutures, annuloplasty rings, stents, staples, valved grafts, dermal grafts for wound healing, orthopedic spinal implants, orthopedic pins, intrauterine devices, urinary stents, maxial facial reconstruction plating, dental implants, intraocular lenses, clips, sternal wires, bone, skin, ligaments, tendons, and combination thereof. Percutaneous devices include, without limitation, catheters or various types, cannulas, drainage tubes such as chest tubes, surgical instruments such as forceps, retractors, needles, and gloves, and catheter cuffs. Cutaneous devices include, without limitation, burn dressings, wound dressings and dental hardware, such as bridge supports and bracing components.

An implantable medical device as described above is generally structured from a base metallic or polymeric platform in a solid state format. The composition of the invention, either alone or as an admixture, controls the release of therapeutic agents from the device for local drug delivery applications.

The compounds, compositions, and admixtures of the invention can also be used to deliver a biologically active agent to the surface of a cosmoceutical (e.g., creams, gels, and lotions), to a pellet, e.g, for controlling the proliferation of pests, such as weeds or insects, or to a membrane, for example, for use in a water purification process in which an antibacterial agent is released into the water.

The following examples, as set forth below and as summarized in Table 3. are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

TABLE 3

| Example | Compound | Description |
|---|---|---|
| 1 | 2 | Cipro: Triethylene glycol |
| 2 | 3 | Cipro: Triethylene glycol |
| 3 | 4 | Cipro: Polyethylene glycol methyl ester |
| 4 | 5 | Cipro: Polyethylene glycol |
| 5 | 6 | Cipro: Polyethylene glycol mono methyl ether |
| 6 | 7 | Cipro: Hexane-1,2,3,4,5,6-hexol |
| 7 | 8 | Cipro: Alkoxylated Polyol |
| 8 | 9 | Hydrocortisone: Triethylene glycol |
| 9 | 10 | Cipro: Pentaerythritol ethoxylate |
| 10 | 11 | Cipro: Xylitol |
| 11 | 12 | Ofloxacin: Triethylene glycol |
| 12 | 2 | Acute Systemic Toxicity |
| 13 | 3 | Acute Systemic Toxicity |
| 14 | 2 | Intracutaneous Reactivity |
| 15 | 3 | Intracutaneous Reactivity |
| 16 | 2 | Coating |
| 17 | 3 | Coating |
| 18 | 2 and 3 | Coating |
| 19 | 2 and 3 | Gel Matrix Composition |
| 20 | 2 | Compounding |
| 21 | 2 | Heat Press |
| 22 | 2 | Drug Release in Solution |
| 23 | 3 | Drug Release in Solution |
| 24 | 2 | Accelerated Drug Release |
| 25 | 2 | Drug Release from device prototype |
| 26 | 2 | MIC/MBC |
| 27 | 3 | MIC/MBC |

### Example 1

### Synthesis and Characterization of Compound 2

Ciprofloxacin HCl (1 mol) and trityl chloride (2.2 mols eqv.) were weighed in a flask and stirred in chloroform (1 L) at room temperature under N₂. Triethylamine (3.2 mols eqv.) was added dropwise into the solution and stirred at room temperature under N₂ for 4 hours.

Methanol (500 mL) was added into the reaction flask and heated to 50° C. for 1.5 hours under N₂. At the end of 1.5 hour reaction, the reaction flask was cooled to room temperature. The resulting solution was washed with water (2×2 L). The organic layer was dried over sodium sulphate. A small amount of methanol was added into solution and the reaction flask was placed in refrigerator overnight. The product was collected by filtration (Compound 1).

Compound 1 (2.1 mol) and DMAP (1.05 eqv.) were weighed in a flask and stirred in anhydrous dichloromethane (900 mL) under N₂ at room temperature until dissolved. Triethylene glycol (1 mol eqv.) was added dropwise into reaction flask. The reaction flask was then placed in an ice bath and solution was stirred under N₂. EDC (8.4 mol eqv.) was weighed and quickly added into the reaction flask. The reaction was allowed to proceed for 1 week at room temperature under N₂. At the end of reaction period, solvent was removed to one third of the original volume by rotary evaporator. Methanol was added into the flask and placed in −20° C. freezer overnight to precipitate. Solution mixture was then filtered, and solid product was collected and dried.

Solid (1 mol) was weighed in a beaker, dichloromethane was added (100 mL), and stirred. Trifluoroacetic acid solution was prepared in water at 3.08 g/mL. Trifluoroacetic acid solution (4 mol eqv.) was added into the beaker dropwise and let stirred for few hours at room temperature. Solution mixture was then filtered and solid product was washed two times with chloroform.

In a beaker, solid was weighed, chloroform: water mixture (1.3:1 v/v) was added into the beaker, and stirred at room temperature. Saturated bicarbonate solution was prepared in water and added to solution mixture dropwise until pH 8 was reached. When desired pH is reached, solution mixture was filtered and solid was collected and dried in vacuum oven for 2 days.

Compound 2: HPLC (mobile phase H2O/TFA and MeCN/TFA) 19.857 min. Sodium analysis=1240 ppm. ¹H NMR (300 MHz, dDMSO) δ (ppm) 1.06-1.22 (CH₂—CH, ciprofloxacin), 3.32 (CH₂—NH, ciprofloxacin), 3.41 (CH₂—N—, ciprofloxacin), 3.56 (CH—, ciprofloxacin), 3.64 (O—CH₂—CH₂—O, TEG), 3.72 (CH₂—O, TEG), 4.24 (CH₂—OOC, TEG), 7.33 (HC═C—N, ciprofloxacin), 7.49 (HC═C—F, ciprofloxacin), 8.31 (N—C(H)═C(CO)—COO—, ciprofloxacin). ¹⁹F NMR (300 MHz, dDMSO) δ (ppm) −124.8 (HC═C—F, ciprofloxacin)

### Example 2

### Synthesis and Characterization of Compound 3

Compound 1 (1 mol) and DMAP (0.505 mol eqv.) were weighed in a flask and stirred in anhydrous dichloromethane (900 mL) under N₂ at room temperature until dissolved. Triethylene glycol (10 mol eqv.) was added dropwise into reaction flask. The reaction flask was placed in an ice bath and solution was stirred under N₂. EDC (4.1 mol eqv.) was weighed and quickly added into the reaction flask. The reaction was allowed to proceed for 1 week at room temperature under N₂. At the end of reaction period, solvent was removed to one third the original volume by rotary evaporator. Methanol was added into the flask and placed in −20° C. freezer overnight to precipitate. Solution mixture was then filtered, and solid product was collected and dried.

Solid was dissolved in chloroform (1.5% w/v) and loaded onto silica resin column packed in chloroform (50:1 w/w silica:solid product). Impurities were eluted through the column using chloroform as mobile phase and then mobile phase was switched to 5% methanol in chloroform to elute product. Fractions corresponding to product were collected and solvent was removed completely by rotary evaporator to give solid product.

Solid (1 mol) was weighed in a beaker, dichloromethane was added (20 mL), and mixture was stirred. Trifluoroacetic acid solution was prepared in water at 3.08 g/mL. Trifluoroacetic acid solution (2 mol eqv.) was added into the beaker dropwise and solution was stirred for 0.5-1 hour at room temperature. Water (40 ml) was added to solution, mixed well, and the aqueous phase was collected. The water extraction was repeated on organic phase and the two aqueous phases were combined. Saturated bicarbonate solution was prepared in water and added to solution mixture dropwise until pH 8 was reached. When desired pH was reached, solution was frozen at −20° C. and product was recovered by lyophilization.

Compound 3: HPLC (mobile phase H2O/TFA and MeCN/TFA) 19.090 min. Sodium analysis=1870 ppm. Mass spectroscopy (m/z) 464.2. ¹H NMR (300 MHz, dDMSO) δ (ppm) 1.06-1.24 (CH₂—CH, ciprofloxacin), 3.14 (CH₂—CH₂—O—CH₂—CH₂, TEG), 3.30 (CH₂—NH, ciprofloxacin), 3.41 (CH₂—N—, ciprofloxacin), 3.56 (CH— and CH₂—OH, ciprofloxacin and TEG, respectively), 3.68 (O—CH₂—CH₂—O and CH₂—O, TEG), 4.27 (CH₂—OOC, TEG), 7.42 (HC═C—N, ciprofloxacin), 7.77 (HC═C—F, ciprofloxacin), 8.43 (N—C(H)═C(CO)—COO—, ciprofloxacin). ¹⁹F NMR (300 MHz, dDMSO) δ (ppm) −124.5 (HC═C—F, ciprofloxacin).

### Example 3

### Synthesis and Characterization of Compound 4

Compound 1 (1.1 mol) and DMAP (0.53 mol eqv.) was weighed in a flask and stirred in anhydrous dichloromethane (870 mL) under N₂ at room temperature until dissolved. Poly(ethylene glycol) methyl ester or Poly(ethylene glycol) methyl ether (1 mol eqv.) was dissolved in dichloromethane (30 mL) and added dropwise into reaction flask. The reaction flask was then placed in an ice bath and solution was stirred under N₂. EDC (4.1 mol eqv.) was weighed and quickly added into the reaction flask. The reaction was allowed to proceed for 10 days at room temperature under N₂. At the end of reaction period, solvent was removed to one third of the original volume by rotary evaporator. Methanol was added into the flask and placed in −20° C. freezer overnight to precipitate. Solution mixture was then filtered, and solid product was collected and dried.

Solid (1 mol) was weighed in a beaker, dichloromethane was added (100 mL), and stirred. Trifluoroacetic acid solution was prepared in water at 3.08 g/mL. Trifluoroacetic acid solution (2 mol eqv.) was added into the beaker dropwise and let stirred for few hours at room temperature. Solution mixture was then filtered and solid product was washed three times with dichloromethane.

In a beaker, solid was weighed, dichloromethane: water mixture (5:1 v/v) is added into the beaker, and stirred at room temperature. Saturated bicarbonate solution was prepared in water and added to solution mixture dropwise until pH 8 is reached. When desired pH was reached, solution mixture was filtered and solid was collected and dried in vacuum oven for 2 days.

Characterization was completed using TLC, HPLC, ¹H NMR analysis.

### Example 4

### Synthesis and Characterization of Compound 5

Compound 1 (2.1 mol) and DMAP (1.05 eqv.) were weighed in a flask and stirred in anhydrous dichloromethane (870 mL) under N₂ at room temperature until dissolved. Poly(ethylene glycol) (1 mol eqv.) was dissolved in dichloromethane (30 mL) and added dropwise into reaction flask. The reaction flask was then placed in an ice bath and solution is stirred under N₂. EDC (8.4 mol eqv.) was weighed and quickly added into the reaction flask. The reaction was allowed to proceed for 10 days at room temperature under N₂. At the end of reaction period, solvent was removed to one third of the original volume by rotary evaporator. Methanol was added into the flask and placed in −20° C. freezer overnight to precipitate. Solution mixture was then filtered, and solid product was collected and dried.

Solid (1 mol) was weighed in a beaker, dichloromethane was added (100 mL), and stirred. Trifluoroacetic acid solution was prepared in water at 3.08 g/mL. Trifluoroacetic acid solution (4 mol eqv.) was added into the beaker dropwise and let stirred for few hours at room temperature. Solution mixture was then filtered and solid product was washed three times with dichloromethane.

In a beaker, solid was weighed, dichloromethane: water mixture (5:1 v/v) was added into the beaker, and stirred at room temperature. Saturated bicarbonate solution was prepared in water and added to solution mixture dropwise until pH 8 is reached. When desired pH was reached, solution mixture was filtered and solid was collected and dried in vacuum oven for 2 days.

Characterization was completed using TLC, HPLC, ¹H NMR analysis.

### Example 5

### Synthesis and Characterization of Compound 6

Compound 1 (2.1 mol) and DMAP (1.05 eqv.) were weighed in a flask and stirred in anhydrous dichloromethane (850 mL) under N₂ at room temperature until dissolved. Polyethylene glycol mono methyl ether (1 mol eqv.) was dissolved in dichloromethane (50 mL) and added into reaction flask dropwise. The reaction flask was then placed in an ice bath and solution is stirred under N₂. EDC (8.4 mol eqv.) was weighed and quickly added into the reaction flask. The reaction was allowed to proceed for 10 days at room temperature under N₂. At the end of reaction period, solvent was removed to one third of the original volume by rotary evaporator. Methanol was added into the flask and placed in −20° C. freezer overnight to precipitate. Solution mixture was then filtered, and solid product was collected and dried.

Solid (1 mol) was weighed in a beaker, dichloromethane was added (100 mL), and stirred. Trifluoroacetic acid solution was prepared in water at 3.08 g/mL. Trifluoroacetic acid solution (4 mol eqv.) was added into the beaker dropwise and let stirred for few hours at room temperature. Solution mixture was then filtered and solid product was washed three times with dichloromethane.

In a beaker, solid was weighed, dichloromethane: water mixture (5:1 v/v) was added into the beaker, and stirred at room temperature. Saturated bicarbonate solution was prepared in water and added to solution mixture dropwise until pH 8 was reached. When desired pH was reached, solution mixture was filtered and solid was collected and dried in vacuum oven for 2 days.

Characterization was completed using TLC, HPLC, ¹H NMR analysis.

### Example 6

### Synthesis and Characterization of Compound 7

Compound 1 (6.1 mol) and DMAP (3.2 mol eqv.) were weighed in a flask and stirred in anhydrous dichloromethane (900 mL) under N₂ at room temperature until dissolved. Hexane-1,2,3,4,5,6-hexol (1 mol eqv.) was added dropwise into reaction flask. The reaction flask was then placed in an ice bath and solution is stirred under N₂. EDC (24.4 mol eqv.) was weighed and quickly added into the reaction flask. The reaction was allowed to proceed for 10 days at room temperature under N₂. At the end of reaction period, solvent was removed to one third of the original volume by rotary evaporator. Methanol was added into the flask and placed in −20° C. freezer overnight to precipitate. Solution mixture was then filtered, and solid product was collected and dried.

Solid (1 mol) was weighed in a beaker, dichloromethane was added (100 mL), and stirred. Trifluoroacetic acid solution was prepared in water at 3.08 g/mL. Trifluoroacetic acid solution (12 mol eqv.) was added into the beaker dropwise and let stirred for few hours at room temperature. Solution mixture was then filtered and solid product was washed three times with dichloromethane.

In a beaker, solid was weighed, dichloromethane: water mixture (5:1 v/v) was added into the beaker, and stirred at room temperature. Saturated bicarbonate solution was prepared in water and added to solution mixture dropwise until pH 8 was reached. When desired pH is reached, solution mixture was filtered and solid was collected and dried in vacuum oven for 2 days.

Characterization was completed using TLC, HPLC, ¹H NMR analysis.

### Example 7

### Synthesis and Characterization of Compound 8

Compound 1 (3.1 mol) and DMAP (1.58 mol eqv.) were weighed in a flask and stirred in anhydrous dichloromethane (900 mL) under N₂ at room temperature until dissolved. Alkoxylated Polyol (1 mol eqv.) was added dropwise into reaction flask. The reaction flask was then placed in an ice bath and solution was stirred under N₂. EDC (12.4 mol eqv.) was weighed and quickly added into the reaction flask. The reaction was allowed to proceed for 10 days at room temperature under N₂. At the end of reaction period, solvent was removed to one third of the original volume by rotary evaporator. Methanol was added into the flask and placed in −20° C. freezer overnight to precipitate. Solution mixture was then filtered, and solid product was collected and dried.

Solid (1 mol) was weighed in a beaker, dichloromethane was added (100 mL), and stirred. Trifluoroacetic acid solution was prepared in water at 3.08 g/mL. Trifluoroacetic acid solution (6 mol eqv.) was added into the beaker dropwise and let stirred for few hours at room temperature. Solution mixture was then filtered and solid product was washed three times with dichloromethane.

In a beaker, solid was weighed, dichloromethane: water mixture (5:1 v/v) was added into the beaker, and stirred at room temperature. Saturated bicarbonate solution was prepared in water and added to solution mixture dropwise until pH 8 was reached. When desired pH is reached, solution mixture is filtered and solid was collected and dried in vacuum oven for 2 days.

Characterization was completed using TLC, HPLC, ¹H NMR analysis.

### Example 8

### Synthesis and Characterization of Compound 9

Hydrocortisone (1 mol) and triethyleneamine (0.5 mols eqv.) were weighed in a flask and stirred in dichloromethane at room temperature under N2. Bis activated carbonate (2 mols eqv.) was added into the solution and stirred at room temperature under N2 overnight. Purification was performed using crystallization and column chromatography.

Characterization was completed using TLC, HPLC, ¹H NMR analysis.

### Example 9

### Synthesis and Characterization of Compound 10

Compound 1 (4.1 mol) and DMAP (2.1 mol eqv.) were weighed in a flask and stirred in anhydrous dichloromethane (870 mL) under N₂ at room temperature until dissolved. Pentaerythritol ethoxylate (1 mol eqv.) was dissolved in dichloromethane (30 mL) and added dropwise into reaction flask. The reaction flask was then placed in an ice bath and solution was stirred under N₂. EDC (16.4 mol eqv.) was weighed and quickly added into the reaction flask. The reaction was allowed to proceed for 10 days at room temperature under N₂. At the end of reaction period, solvent was removed to one third of the original volume by rotary evaporator. Methanol was added into the flask and placed in −20° C. freezer overnight to precipitate. Solution mixture was then filtered, and solid product was collected and dried.

Solid (1 mol) was weighed in a beaker, dichloromethane was added (100 mL), and stirred. Trifluoroacetic acid solution was prepared in water at 3.08 g/mL. Trifluoroacetic acid solution (8 mol eqv.) was added into the beaker dropwise and let stirred for few hours at room temperature. Solution mixture was then filtered and solid product was washed three times with dichloromethane.

In a beaker, solid was weighed, dichloromethane: water mixture (5:1 v/v) was added into the beaker, and stirred at room temperature. Saturated bicarbonate solution was prepared in water and added to solution mixture dropwise until pH 8 was reached. When desired pH was reached, solution mixture was filtered and solid was collected and dried in vacuum oven for 2 days.

Characterization was completed using TLC, HPLC, ¹H NMR analysis.

### Example 10

### Synthesis and Characterization of Compound 11

Compound 1 (5.1 mol) and DMAP (2.63 mol eqv.) are weighed in a flask and stirred in anhydrous dichloromethane (870 mL) under N₂ at room temperature until dissolved. Xylitol (1 mol eqv.) is added dropwise into reaction flask. The reaction flask is then placed in an ice bath and solution is stirred under N₂. EDC (20.4 mol eqv.) is weighed and quickly added into the reaction flask. The reaction is allowed to proceed for 10 days at room temperature under N₂. At the end of reaction period, solvent is removed to one third of the original volume by rotary evaporator. Methanol is added into the flask and placed in −20° C. freezer overnight to precipitate. Solution mixture is then filtered, and solid product is collected and dried.

Solid (1 mol) is weighed in a beaker, dichloromethane is added (100 mL), and stirred. Trifluoroacetic acid solution is prepared in water at 3.08 g/mL. Trifluoroacetic acid solution (10 mol eqv.) is added into the beaker dropwise and let stirred for few hours at room temperature. Solution mixture is then filtered and solid product is washed three times with dichloromethane.

In a beaker, solid is weighed, dichloromethane: water mixture (5:1 v/v) is added into the beaker, and stirred at room temperature. Saturated bicarbonate solution is prepared in water and added to solution mixture dropwise until pH 8 is reached. When desired pH is reached, solution mixture is filtered and solid is collected and dried in vacuum oven for 2 days.

Characterization are performed using TLC, HPLC, ¹H NMR analysis.

### Example 11

### Synthesis and Characterization of Compound 12

Ofloxacin (2.1 mol) and DMAP (1.05 eqv.) were weighed in a flask and stirred in anhydrous dichloromethane (900 mL) under N2 at room temperature until dissolved. Triethylene glycol (1 mol eqv.) was added into reaction flask. The reaction flask was then placed in an ice bath and solution was stirred under N2. EDC (8.4 mol eqv.) was weighed and quickly added into the reaction flask. The reaction was allowed to proceed for 1 week at room temperature under N2. The resulting solution was washed with water (2×2 L). The organic layer was dried over sodium sulphate. Mixture solution was filtered and filtrate was collected. Dichloromethane was removed to approximately 20% of the original volume by rotary evaporator. Acetone was added into the flask at 1:1 (v/v) ratio and placed in −20° C. freezer overnight to precipitate. Precipitate was then filtered, collected and dried.

Compound 12: HPLC (mobile phase H2O/TFA and MeCN/TFA) 19.678 min and 19.868 min. Mass spectroscopy (m/z) 836.4. 1H NMR (300 MHz, CDCl3) δ (ppm) 1.56 (CH3-CH, ofloxacin), 2.37 (CH3-N, ofloxacin), 2.56 (—O—CH2-CH (CH3), ofloxacin), 3.34 (—N—CH2-CH2-N—, ofloxacin), 3.77 (O—CH2-CH2-O, TEG), 3.85 (—CH2-O, TEG), 4.38 (CH—C(CH3), ofloxacin), 4.84 (CH2-OOC, TEG), 7.21 (HC═C—F, ofloxacin), 8.22 (N—C(H)═C(CO)—COO—, ofloxacin).

### Example 12

### Acute Systemic Toxicity Testing of Compound 2

Compound 2 was dissolved in PBS (4×10⁻⁵-9.7×10⁻¹ mg/mL) and tested for Acute Systemic Toxicity following ISO 10993-11. A single injection dose of 50 mL/kg per mouse (˜1 mL) was administered to 5 mice per test sample. Mice were observed immediately post injection and at 4, 24, 48 and 72 h for signs of toxicity compared to control. Higher dosage demonstrated no signs of toxicity.

### Example 13

### Acute Systemic Toxicity Testing of Compound 3

Compound 3 was dissolved in PBS (8×10⁻⁴-8×10⁻² mg/mL) and tested for Acute Systemic Toxicity following ISO 10993-11. A single injection dose of 50 mL/kg per mouse (˜1 mL) was administered to 5 mice per test sample. Mice were observed immediately post injection and at 4, 24, 48 and 72 h for signs of toxicity compared to control. Compound 3 at all concentrations showed no signs of toxicity.

### Example 14

### Intracutaneous Reactivity Testing of Compound 2

Compound 2 was dissolved in PBS (9.2×10 mg/mL) and tested for Intracutaneous Reactivity in accordance with ISO 10993-10: 2010 Standard, Biological Evaluation of Medical Devices, Part 10: Tests for Irritation and Skin Sensitization, Pages 11-14. Each rabbit received five sequential 0.2 mL intracutaneous injections along either side of the dorsal mid-line with the test article on one side and the control on the other. Three New Zealand rabbits were used per sample and control. The injection sites were observed and scored for erythema (redness) and edema (swelling) after 24, 48, and 72 h on a scale of 1 to 4. Compound 2 showed no signs of irritation and was deemed a non-irritant.

### Example 15

### Intracutaneous Reactivity Testing of Compound 3

Compound 3 was dissolved in PBS (5.4×10⁻² mg/mL) and tested for Intracutaneous Reactivity in accordance with ISO 10993-10: 2010 Standard, Biological Evaluation of Medical Devices, Part 10: Tests for Irritation and Skin Sensitization, Pages 11-14. Each rabbit received five sequential 0.2 mL intracutaneous injections along either side of the dorsal mid-line with the test article on one side and the control on the other. Three New Zealand rabbits were used per sample and control. The injection sites were observed and scored for erythema (redness) and edema (swelling) after 24, 48, and 72 h on a scale of 1 to 4. Compound 3 showed no signs of irritation and was deemed a non-irritant.

### Example 16

### Coating Compound 2 on Polymeric Surfaces

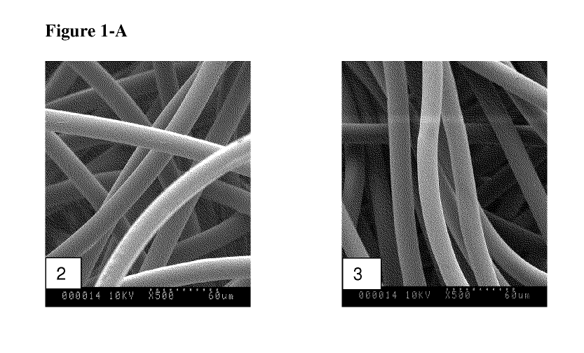

Dacron meshes (TDA PETNF203) at 0.5 cm×2 cm and Hernia meshes were dip coated with a range of Compound 2 solutions (1-30 mg/mL) in various solvents (DMF, DMSO, Methanol). Further increases in loading (up to ˜13 mg) were achieved by dipping the Dacron meshes in solution multiple times at 30 mg/ml with drying periods between each dip. Loading was determined by stripping samples for 6 h in DMF and analyzing by RP-HPLC using established protocols. SEM analysis of coated meshes in DMF showed a smooth coating with limited webbing (FIG. 1). No changes in chemical structure were observed after stripping coated sample in d₆-DMSO for 1 h and analyzing by ¹H NMR.

Dacron meshes were also coated with Compound 2 and Chlrohexidine (CHX) in various solvents.

SEM analysis of coated meshes showed a smooth coating. The release profile and biological efficacy of Compound 2 (Cip) was not impacted by the presence of CHX.

### Example 17

### Coating Compound 3 on Polymeric Surfaces

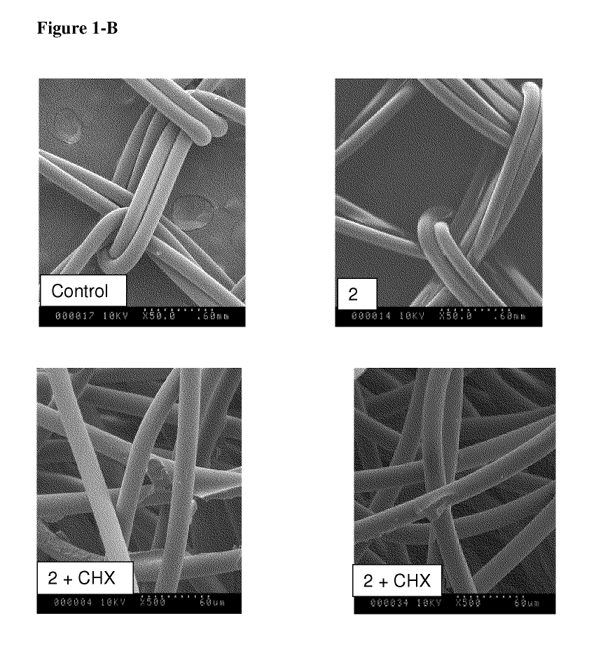

Dacron mesh (TDA PETNF203) at 0.5 cm×2 cm was dip coated with Compound 3 and dried at room temperature under vacuum. SEM of coated mesh showed a smooth coating with limited webbing (FIG. 2).

### Example 18

### Coating Compound 2 and 3 on Metallic Surfaces

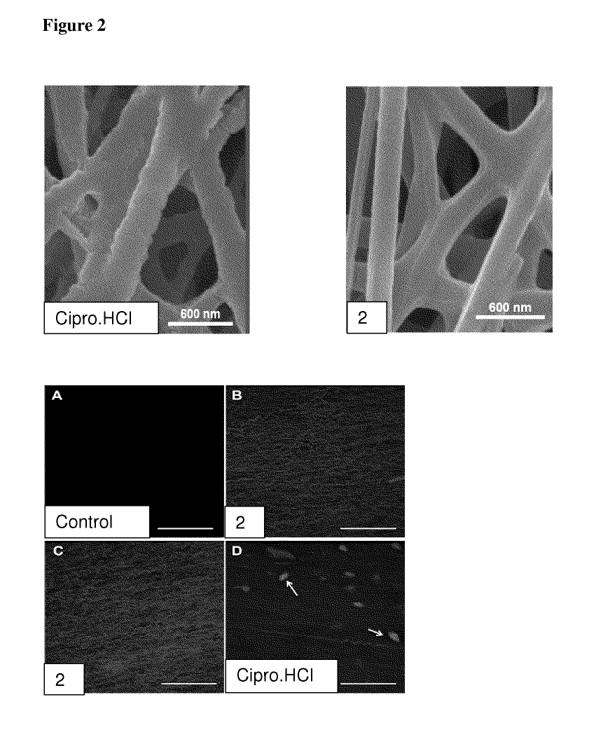

Stainless steel coupons and orthopedic screws were dipped once for 30s in either a 10 mg/mL solution of Compound 2, Compound 3, ciprofloxacin HCl in DMF, or DMF alone as control. Compound 2 and ciprofloxacin HCl samples were dried in a 50° C. flow oven for 5 h while Compound 3 was dried at 60° C. After drying, visual observations were made using light microscopy (FIG. 3). Coupons with ciprofloxacin HCl had a white uneven coating, while those coated with Compounds 2 and 3 were clear.

### Example 19

### Incorporating Compounds 2 and 3 into Gel Matrix

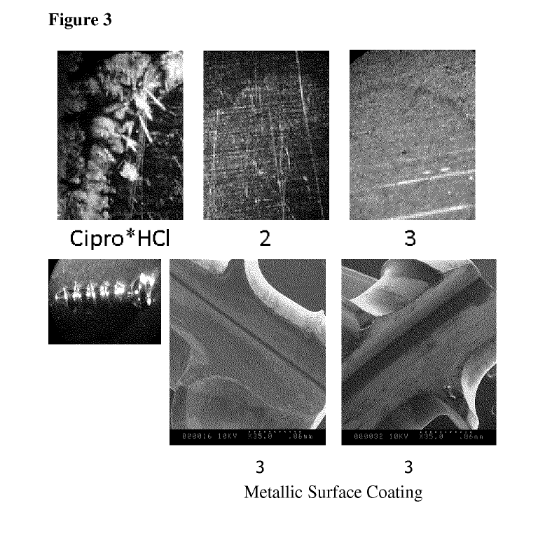

A 3% alginate solution in water was made and added to ciprofloxacin HCl, Compound 2 and Compound 3 at 25 mg/mL and stirred overnight. Solutions were added to a 10 mM solution of CaSO₄ dropwise for 5 min to crosslink. Gels were removed from CaSO₄ solution and visual observations made using light microscopy (FIG. 4). Gels with Compounds 2 and 3 were clear similar to alginate alone while gels with ciprofloxacin HCl were opaque.

### Example 20

### Compounding with Compound 2

Compound 2, ciprofloxacin HCl were compounded into extruded rods with various base polymers (SIBS, Carbothane, PE, PVC) at 2 and 5 wt %. All compounding was done with a DSM Xplore 15 mL micro-compounder. Processing parameters were adjusted according to base polymer. Drug release in 37° C. PBS was monitored up to 30d. drug released from 5% Compound 2+Carbothane rods was measured by RP-HPLC using established protocols: 1d=302 ng/mL, 10d=1748 ng/mL, 30d=10006 ng/mL.

### Example 21

### Compatibility of Compound 2 with Various Base Polymers

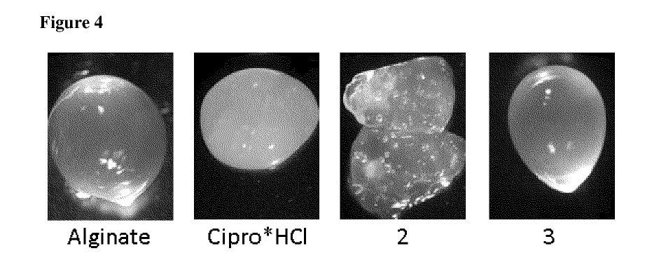

Various base polymers were tested: SIBS, Tecoflex, Tecoflex+30% Ba₂SO₄, Carbothane 95A, and Carbothane 95 A+30% Ba₂SO₄. Base polymer (4 g) and cipro (control) or Compound 2 (2 wt %) were weighed in a vial. Appropriate solvent was added into the vial to allow surface coating of base polymer beads. Solvent was removed and coated base polymer beads were melted at 170° C. for 4 minutes, pressed at 1 ton pressure for 1 minute, and quenched in cold water. Visual appearance of each film was observed and noted. Films prepared by blending base polymer and cipro demonstrated phase separation and heterogeneous morphology. Films prepared by blending base polymer and Compound 2 demonstrated a homogenous morphology (FIG. 5).

### Example 22

### Drug Release from Compound 2 in Solution

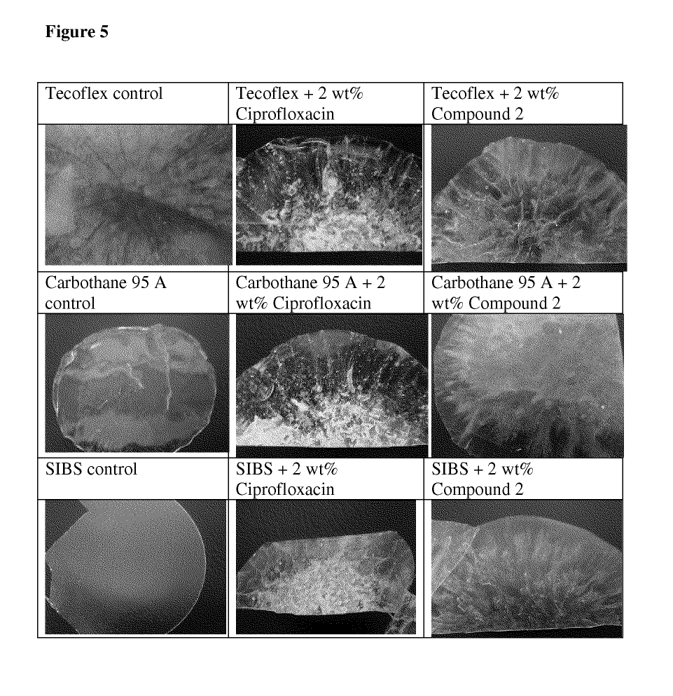

Compound 2 was dissolved in PBS (pH 7.4) at 0.1 mg/ml and placed in incubator at 37° C. At each time point (0, 1, 3, 7, 14, 21, & 28 days), solution was removed from incubator and analyzed for the drug by RP-HPLC using established protocols (FIG. 6). After 28 days, ˜8% total drug was released from Compound 2, demonstrating slow and sustained release under these conditions. The release profile of compound 2 was also evaluated in a device prototype assembly using bovine serum, blood (porcine) or a gel matrix.

### Example 23

### Drug Release from Compound 3 in Solution

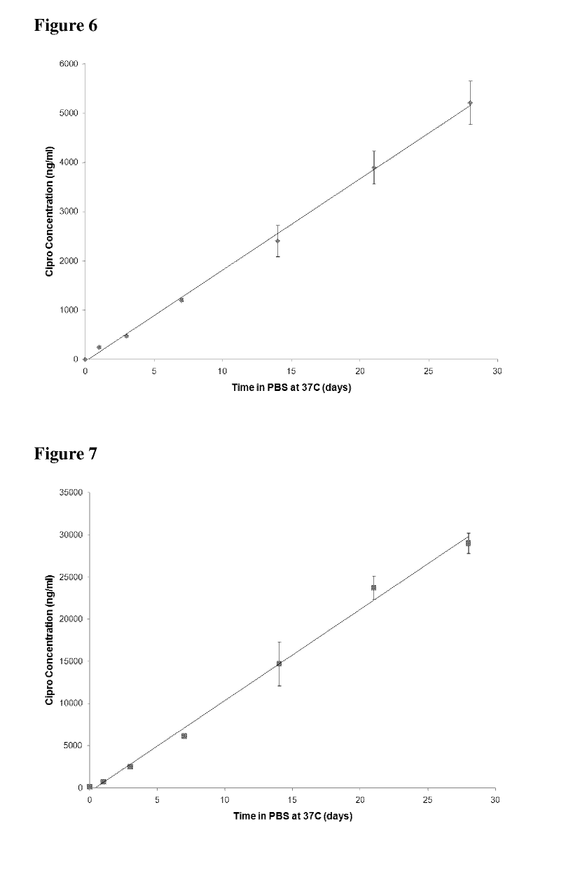

Compound 3 was dissolved in PBS (pH 7.4) at 0.1 mg/ml and placed in incubator at 37° C. At each time point (0, 1, 3, 7, 14, 21, & 28 days), solution was removed from incubator and analyzed for drug by RP-HPLC using established protocols. A linear increase in the drug concentration was observed with time out to at least 28 days (FIG. 7).

### Example 24

### Accelerated Drug Release from Compound 2

Compound 2 was prepared at 10 mg/ml in 0.1 N HCl (final pH ˜4), 0.1 N NaOH (final pH ˜10), and PBS (final pH ˜7). Samples were incubated at 37° C. in acidic, basic, or neutral conditions. At each time point, the drug concentration was quantified by RP-HPLC. Faster drug release was demonstrated under acidic and basic conditions: basic pH (100% release in less than 1 day)<<acidic pH (˜71% after 7 days)<<neutral pH (˜2% after 7 days).

### Example 25

### Drug Release from Compound 2 Coated onto Dacron

Compound 2 was coated onto 0.5 cm×2 cm Dacron meshes and dried at 60° C. overnight. Coated meshes were assembled on catheters. Drug release from the coated meshes pre- and post-assembly was carried out in 2 ml PBS (pH 7.4) at 37° C. for 24 h. drug released into solution was quantified by RP-HPLC.

### Example 26

### MIC & MBC Determination for Compound 2 and Drug Released from Compound 2

The antimicrobial efficacy of Compound 2 and drug released from Compound 2 was investigated using a standard broth microdilution method. The minimum inhibitory concentration (MIC) and minimum bactericidal concentration (MBC) were investigated for Compound 2, drug released from Compound 2, triethylene glycol (TEG), Ciprofloxacin hydrochloride (Cipro®HCl), chlorhexidine diacetate (CHX-A), and Compound 2+CHX-A. The MIC is defined as the lowest concentration of an antimicrobial agent that will inhibit visible growth of a microorganism after overnight incubation. The MBC is defined as the lowest concentration of an antimicrobial agent required to kill 99.9% of a microorganism population. This study was carried out using two gram positive bacteria, E. faecalis (ATCC 29212) and S. aureus (ATCC 25923), and two gram negative bacteria, E. coli (ATCC 25922) and P. aeruginosa (ATCC 27853). The test samples were prepared using 2-fold dilutions per well and covered a concentration range at least 2 dilutions above and 2 dilutions below literature MIC values for the microorganisms tested. Table 4 summarizes the results of the study for S. aureus. Compound 2 did not show any antimicrobial activity at the highest concentration tested. Drug released from Compound 2 showed antimicrobial activity consistent with both the Cipro®HCl controls and literature values. No antimicrobial activity was observed with TEG linker at the highest concentration tested. Testing Compound 2 in combination with CHX-A did not affect the activity of CHX-A, demonstrating the potential for additive or combination therapy with a second antimicrobial agent. Similar results were observed for the other microorganisms tested.

TABLE 4
Experimental and literature MIC & MBC values for Compound 2 against S. aureus

| | | Tested | | | | |
|---|---|---|---|---|---|---|
| | | Concentration | MIC (ug/ml) | | MBC (ug/ml) | |
| Samples | | Range (ug/ml) | Experimental | Literature | Experimental | Literature |
| Compound 2 | | 78-0.076 | >78 | n/d | >78 | n/d |
| TEG | | 100-0.098 | >100 | | >100 | |
| Cipro*HCl | | 4-0.004 | 0.25-0.5 | 0.5 | 0.5-1 | 0.9 |
| Drug Released from Compound 2 | | 4-0.004 | 0.5 | | 0.5 | |
| CHX-A | | 128-0.125 | 1 | 0.9 | 1-4 | 3.9 |
| Compound 2 + CHX-A | 2 | 78-0.076 | — | | — | |
| | CHX-A | 128-0.125 | 1 | | 2 | |

### Example 27

### MIC & MBC Determination for Compound 3 and Drug Released from Compound 3

The antimicrobial efficacy of Compound 3 and drug released from Compound 3 investigated using a standard broth microdilution method. The MIC and MBC were investigated for Compound 3, drug released from Compound 3, TEG, Cipro®HCl, CHX-A, and Compound 3+CHX-A. This study was carried out using two gram positive bacteria, E. faecalis (ATCC 29212) and S. aureus (ATCC 25923), and two gram negative bacteria, E. coli (ATCC 25922) and P. aeruginosa (ATCC 27853). The test samples were prepared using 2-fold dilutions per well and covered a concentration range at least 2 dilutions above and 2 dilutions below literature MIC values for the microorganisms tested. Table 5 summarizes the results of the study for S. aureus. Compound 3 did not show any antimicrobial activity at the highest concentration tested. Drug released from compound 3 showed antimicrobial activity consistent with both the Cipro®HCl controls and literature values. Testing Compound 3 in combination with CHX-A did not affect the activity of CHX-A, demonstrating the potential for additive or combination therapy with a second antimicrobial agent. Similar results were observed for the other microorganisms tested.

TABLE 5
Experimental and literature MIC & MBC values for Compound 3 against S. aureus

| | | Tested | | | | |
|---|---|---|---|---|---|---|
| | | Concentration | MIC | | MBC (ug/ml) | |
| Samples | | Range (ug/ml) | Experimental | Literature | Experimental | Literature |
| Compound 3 | | 55.5-0.054 | >55.5 | n/d | >55.5 | n/d |
| TEG | | 100-0.098 | >100 | | >100 | |
| Cipro*HCl | | 4-0.004 | 0.25-0.5 | 0.5 | 0.5-1 | 0.9 |
| Cipro Released from Compound 3 | | 4-0.004 | 0.5 | | 1 | |
| CHX-A | | 128-0.125 | 1 | 0.9 | 1-4 | 3.9 |
| Compound 3 + CHX-A | 3 | 55.5-0.054 | — | | — | |
| | CHX-A | 128-0.125 | 1 | | 8 | |

All publications, patent applications, and patents mentioned in this specification are herein incorporated by reference.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments.

What is claimed is:

1. An article comprising a coated surface, wherein said coated surface comprises a compound having a structure according to formula (I):
Bio¹-Link¹-(Bio²-R¹)ₘ (I),
or a pharmaceutically acceptable salt thereof, wherein
Bio¹ is formed from a biologically active agent;
m is 1, 2, 3, 4, or 5;
each Bio² is absent or independently formed from a biologically active agent, and wherein each Bio², when present, comprises a covalent bond to Link¹;
R¹ is present only when Bio² is absent and is a terminal group selected from the group consisting of H, OH, optionally substituted C1-C6 alkyl, and optionally substituted C1-C6 alkoxy;
Link¹ is an oligomeric organic, organosilicon, or organosulfone segment having a molecular weight between 60 and 2000 Daltons.

2. The article of claim 1, wherein said compound has a structure according to formula (I-A),
Bio¹-Link¹-Bio²-R¹ (I-A),
or a pharmaceutically acceptable salt thereof, wherein
Bio¹ is formed from a biologically active agent;
Bio² is absent or formed from a biologically active agent;
R¹, when present, is H, OH, optionally substituted C1-C6 alkyl, or optionally substituted C1-C6 alkoxy; and
Link¹ is an oligomeric organic, organosilicon, or organosulfone segment having a molecular weight between 60 and 2000 Daltons.

3. The article of claim 1 or 2, wherein Bio² is absent.

4. The article of claim 1 or 2, wherein Bio² is present.

5. The article of claim 4, wherein Bio¹ and Bio² are formed from biologically active agents that have the same structure.

6. The article of claim 5, wherein Bio¹ and Bio² are formed from biologically active agents that have different structures.

7. The article of any one of claims 1-6, wherein each Bio¹ and Bio², when present, has a molecular weight ranging from 100 to 1000, from 200 to 1000, from 200 to 900, from 200 to 800, from 200 to 700, from 200 to 600, from 200 to 500, or from 200 to 400 Daltons.

8. The article of any one of claims 1-7, wherein each Bio¹ and Bio², when present, is formed from a biologically active agent selected from the group consisting of: anti-inflammatory agents, anti-thrombotic agents; anti-oxidant agents, anti-coagulant agents, anti-microbial agents, anti-proliferative agents, cell receptor ligands, and bio-adhesive molecules.

9. The article of claim 8, wherein one or both of Bio¹ and Bio², when present, is formed from an anti-microbial agent.

10. The article of claim 8 or 9, wherein one or both of Bio¹ and Bio², when present, is independently, is formed from an antibiotic.

11. The article of claim 10, wherein said antibiotic is a fluoroquinolone antibiotic.

12. The article of claim 11, wherein said antibiotic is selected from the group consisting of: norfloxancin, oflxacin, ciprofloxacin, levofloxacin, moxifloxacin, and gatifloxacin.

13. The article of claim 12, wherein said antibiotic is ciprofloxacin.

14. The article of any one of claims 1-6, wherein one or both of Bio¹ and Bio² is a protein or a peptide.

15. The article of any one of claims 1-14, wherein Link¹ has a molecular weight between 60 and 700 Daltons.

16. The article of any one of claims 1-15, wherein Link¹ is formed from a diol, a diamine, or an α,ω-aminoalcohol.

17. The article of claim 16, wherein Link¹ is formed from a diol.

18. The article of claim 16, wherein Link¹ is formed from a polyethylene oxide having terminal amino or hydroxyl groups, and wherein Link1 comprises 1-3, 1-5, 1-10, or 1-20 ethylene oxide repeating units.

19. The article of claim 16, wherein Link¹ is formed from a compound selected from the group consisting of: ethylene glycol; butane diol; hexane diol; hexamethylene diol; 1,5-pentanediol; 2,2-dimethyl-1,3 propanediol; 1,4-cyclohexane diol; 1,4-cyclohexanedimethanol; tri(ethylene glycol); poly(ethylene glycol), where the molecular weight is between 100 and 2000 Daltons; poly(ethylene oxide) diamine, where the molecular weight is between 100 and 2000 Daltons; lysine esters; silicone diols; silicone diamines; polyether diols; polyether diamines; carbonate diols; carbonate diamines; dihydroxy vinyl derivatives; dihydroxydiphenylsulfone; ethylene diamine; hexamethylene diamine; 1,2-diamino-2-methylpropane; 3,3-diamino-n-methyldipropylamine; 1,4-diaminobutane; 1,7-diaminoheptane; and 1,8-diaminooctane.

20. The article of claim 19, wherein Link¹ is formed from tri(ethylene glycol).

21. The article of any one of claims 1-15, wherein Link¹ is formed from a dicarboxylic compound or a diisocyanate.

22. The article of any one of claims 1-3 and 7-15, wherein Bio² is absent, and Link¹ is formed from a monoalcohol or a monoamine.

23. The article of claim 1 or 2, wherein m is 1, Bio¹ and Bio² are both formed from ciprofloxacin, and Link¹ is formed from tri(ethylene glycol).

24. The article of claim 1 or 2, wherein m is 1, Bio¹ is formed from ciprofloxacin, Bio² is absent, and Link¹ is formed from tri(ethylene glycol).

25. The article of any one of claims 1-24, wherein said coating comprises a second compound having a structure according to formula (I) or formula (I-A), wherein each Bio¹, Link¹, and Bio² is as defined in any of claims 1-19.

26. The article of any one of claims 1-25, wherein said coating is substantially free of any biologically active agent used to form Bio¹ and/or Bio², wherein the biologically active agent is not included in a compound according to formula (I).

27. The article of any one of claims 1-25, wherein said coating further comprises free biologically active agent, wherein the mole ratio of the compound according to formula (I) to the free biologically active agent is from 0.1:1 to 1:0.1.

28. The article of any one of claims 1-27, wherein said compound according to formula (I) has reduced biological activity compared to the biologically active agent used to form Bio¹ and/or Bio².

29. The article of claim 28, wherein said compound according to formula (I) or formula (I-A) has 0%-20% of the biological activity of the biologically active agent used to form Bio¹ and/or Bio².

30. The article of any one of claims 1-29, wherein said coating comprises a pharmaceutically acceptable salt of the compound according to formula (I) or formula (I-A).

31. The article of claim 30, wherein said pharmaceutically acceptable salt is the trifluoroacetate or the hydrochloride salt.

32. The article of any of claims 1-31, wherein said article is a filter, film, fiber, sheet, or an implantable medical device.

33. The article of claim 32, wherein implantable device is selected from: prostheses pacemakers, electrical leads, defibrillators, artificial hearts, ventricular assist devices, anatomical reconstruction prostheses, artificial heart valves, heart valve stents, pericardial patches, surgical patches, coronary stents, vascular grafts, vascular and structural stents, vascular or cardiovascular shunts, biological conduits, pledges, sutures, annuloplasty rings, stents, staples, valved grafts, dermal grafts for wound healing, orthopedic spinal implants, orthopedic devices, ophthalmic implants, intrauterine devices, stents, maxial facial reconstruction plating, dental implants, intraocular lenses, clips, sternal wires, bone, skin, ligaments, sutures, hernia mesh, tendons, and combinations thereof

34. The article of claim 32, wherein
said article is a percutaneous device selected from: catheters, cannulas, drainage tubes, and surgical instruments, or
said article is a cutaneous device selected from burn dressings, wound dressings, and dental hardware.

35. The article of claim 34, wherein said surgical instrument is selected from: forceps, retractors, needles, gloves, and catheter cuffs.

36. The article of claim 35, wherein said article is a catheter cuff.

37. The article of any one of claims 1-36, wherein said coating has a thickness between 0.5 to 120 μM.

38. The article of any one of claims 1-37, wherein said article comprises a fibrous polymer matrix comprising the compound or compounds according to formula (I) and/or formula (I-A).

39. The article of any one of claims 1-31, wherein said article comprises a mixture of two or more compounds according to formula (I) and/or formula (I-A).

40. The article of claim 38, wherein said polymer matrix is formed from a biodegradable polymer.

41. The article of claim 40, wherein said polymer is polylactic acid, polycaprolactone, or polyurethane.

42. The article of claim 38, wherein said polymer matrix is formed from a nonbiodegradable polymer.

43. The article of claim 42, wherein said polymer is poly(ethylene terephthalate).

44. The article of claim 33, wherein said article is a catheter cuff.

45. The article of claim 44, wherein said catheter cuff is a vascular access catheter cuff.

46. The article of claim 43, wherein said article is an orthopedic device.

47. The article of claim 46, wherein said orthopedic device is a wire, pin, rod, nail, screw, disk, plate, bracket, or splint.

48. The article of claim 33, wherein said ophthalmic implant is a punctal plug.

49. A method of preventing infection in a subject in need thereof, said method comprising implanting a device comprising a coated surface, wherein said coated surface comprises a compound having a structure according to formula (I):
Bio¹-Link¹-(Bio²-R¹)ₘ (I),
or a pharmaceutically acceptable salt thereof, wherein
Bio¹ is formed from a biologically active agent;
m is 1, 2, 3, 4, or 5;
each Bio² is absent or independently formed from a biologically active agent, and wherein each Bio², when present, comprises a covalent bond to Link¹;
R¹ is present only when Bio² is absent and is a terminal group selected from the group consisting of H, OH, optionally substituted C1-C6 alkyl, and optionally substituted C1-C6 alkoxy; and
Link¹ is an oligomeric organic, organosilicon, or organosulfone segment having a molecular weight between 60-2000 Daltons.

50. The method of claim 49, wherein said compound has a structure according to formula (I-A),
Bio¹-Link¹-Bio²-R¹ (I-A),
or a pharmaceutically acceptable salt thereof, wherein
Bio¹ is formed from a biologically active agent;
Bio² is absent or formed from a biologically active agent;
R¹, when present, is H, OH, optionally substituted C1-C6 alkyl, or optionally substituted C1-C6 alkoxy; and
Link¹ is an oligomeric organic, organosilicon, or organosulfone segment having a molecular weight between 60-2000 Daltons.

51. The method of claim 49 or 50, wherein said compound is the compound of any one of claims 3-38.

52. An admixture comprising a base polymer and a compound having a structure according to formula (I),
Bio¹-Link¹-(Bio²-R¹)ₘ (I),
or a pharmaceutically acceptable salt thereof, wherein
Bio¹ is formed from a biologically active agent;
m is 1, 2, 3, 4, or 5;
each Bio² is absent or independently formed from a biologically active agent, and wherein each Bio², when present, comprises a covalent bond to Link¹;
R¹ is present only when Bio² is absent and is represents a terminal group selected from the group consisting of H, OH, optionally substituted C1-C6 alkyl, and optionally substituted C1-C6 alkoxy;
Link¹ is an oligomeric organic, organosilicon, or organosulfone segment having a molecular weight between 60 and 2000 Daltons.

53. The admixture of claim 52, wherein said compound has a structure according to formula (I-A),
Bio¹-Link¹-Bio²-R¹ (I-A),
or a pharmaceutically acceptable salt thereof, wherein
Bio¹ is formed from a biologically active agent;
Bio² is absent or formed from a biologically active agent;
R¹, when present, is H, OH, optionally substituted C1-C6 alkyl, or optionally substituted C1-C6 alkoxy; and
Link¹ is an oligomeric organic, organosilicon, or organosulfone segment having a molecular weight between 60 and 2000 Daltons.

54. The admixture of claim 52 or 53, wherein said compound is the compound of any one of claims 3-28.

55. The admixture of any one of claims 52-54, wherein said admixture is a polymer matrix.

56. A method for coating a surface with a composition, said composition comprising:
(a) a compound having a structure according to formula (I),
Bio¹-Link¹-(Bio²-R¹)ₘ (I),
or a pharmaceutically acceptable salt thereof, wherein
Bio¹ is formed from a biologically active agent;
m is 1, 2, 3, 4, or 5;
each Bio² is absent or independently formed from a biologically active agent, and wherein each Bio², when present, comprises a covalent bond to Link¹;
R¹ is present only when Bio² is absent and is represents a terminal group selected from H, OH, optionally substituted C1-C6 alkyl, or optionally substituted 01-C6 alkoxy;
Link¹ is an oligomeric organic, organosilicon, or organosulfone segment having a molecular weight between 60 and 2000 Daltons
and
(b) a suitable medium in which the compound of (a) is soluble; and
said method comprising contacting the surface with said composition.

57. The method of claim 56, wherein said compound has a structure according to formula (I-A),
Bio¹-Link¹-Bio²-R¹ (I-A),
or a pharmaceutically acceptable salt thereof, wherein
Bio¹ is formed from a biologically active agent;
Bio² is absent or formed from a biologically active agent;
R¹, when present, is H, OH, optionally substituted C1-C6 alkyl, or optionally substituted C1-C6 alkoxy; and
Link¹ is an oligomeric organic, organosilicon, or organosulfone segment having a molecular weight between 60 and 2000 Daltons.

58. The method of claim 56 or 57, wherein said compound is as set forth in any of claims 3-28.

59. The method of any one of claims 56-58, wherein (b) is an organic solvent or aqueous solvent.

60. The method of claim 59, wherein said polar organic solvent is tetrahydrofuran or N,N-dimethylformamide.

61. The method of claim 59 or 60, wherein the concentration of (a) is between 0.05-150 mg/mL.

62. The method of claim 59 or 60, wherein said article comprises a mixture of two or more compounds according to formula (I) and/or formula (I-A).

63. The method of claim 59 or 60, wherein one or both of Bio¹ and Bio², when present, is formed from an anti-microbial agent.

64. The method of claim 59 or 60, wherein Bio¹ is formed from a first biologically active agent, and Bio², when present, is formed from a second biologically active agent.

65. The method of claim 64, wherein said first biologically active agent is an antibiotic.

66. The method of claim 64 or 65, wherein said second biologically active agent is an antibiotic.

67. The method of claim 65 or 66, wherein said first antibiotic is a fluoroquinolone antibiotic.

68. The method of any one of claims 65-67, wherein said second antibiotic is a fluoroquinolone antibiotic.

69. The method of any one of claims 66-68, wherein said first antiobiotic is same as said second antibiotic.

70. The method of claim any one of claims 67-69, wherein said antibiotic is selected from the group consisting of: norfloxancin, oflxacin, ciprofloxacin, levofloxacin, moxifloxacin, and gatifloxacin.

71. The method of claim 70, wherein said antibiotic is ciprofloxacin.
